(19) United States
(12) Patent Application Publication
(10) Pub. No.: US 2006/0008422 A1
(43) Pub. Date: Jan. 12, 2006

(54) Dentifrice compositons and abrasive systems

(76) Inventors: Abraham Araya, Warrington (GB); Jonathan Edward Creeth, Weybridge (GB); Ian Patrick McKeown, Warrington (GB); Peter William Stanier, Warrington (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/029,581

(22) Filed: Jan. 5, 2005

Related U.S. Application Data
(60) Provisional application No. 60/535,616, filed on Jan. 9, 2004.

(51) Int. Cl. A61K 8/22; A61K 8/21

(52) U.S. Cl. 424/49

(57) ABSTRACT

A dentifrice composition comprising an abrasive system comprising at least one abrasive silica which may be selected from a first silica having a Radioactive Dental Abrasion (RDA) in the range 30 to 150 and a second silica an RDA in the range 100 to 300, and a crystalline aluminosilicate having an average crystallite size below 0.2 μm plus an orally acceptable carrier. The content by weight of the first silica being greater than that of the second silica and the RDA of the second silica being greater than that of first silica.

### FIELD OF THE INVENTION

This invention relates to dentifrice compositions and abrasive systems for use in high cleaning, controlled abrasivity compositions said abrasive system comprising a combination of amorphous silica and crystalline aluminosilicate.

### BACKGROUND OF THE INVENTION

Dentifrices commonly incorporate an abrasive material for mechanical cleaning and polishing of teeth by physical abrading deposits and they may also include a chemical cleaning agent.

The abrasive material is primarily intended to effect mechanical removal of deposits from the surface of teeth, e.g. removal of pellicle film adhered to the tooth surface. Pellicle film is prone to discolouration and staining, e.g. by comestibles such as tea and coffee, and by tars and particulates in exhaled cigarette smoke, resulting in an unsightly appearance of the teeth. While such mechanical removal is important for effective cleaning, it is vital that the abrasive used is not unduly harsh in order to minimise damage, e.g. scratching, to the teeth.

Synthetically produced amorphous silicas are often the favoured abrasive component in dentifrices and can be readily tailored during the production process to possess predetermined abrasive and other physical characteristics appropriate for use in dentifrices. Precipitated silicas are particularly useful as abrasive components and are generally the material of choice in dentifrice compositions.

Although silicas are particularly effective for mechanical cleaning by abrasion, they make no significant contribution in terms of chemical cleaning.

Crystalline aluminosilicates (zeolites) have been used as cleaning agents in dentifrice compositions. They possess a mechanical cleaning action (abrasivity) and are also known to bind calcium ions. Desirably, a dental cleaning agent combines relatively good cleaning with minimal abrasion of dentine. It has been found that most available zeolites are too abrasive to provide adequate cleaning without unacceptable abrasion.

There remains a need for formulations with improved cleaning without increased abrasivity, beyond what may be achieved with silicas alone. Surprisingly, it has now been found that the use of a combination of a particular silica or silicas with a specific aluminosilicate can result in a dentifrice composition having good cleaning with acceptable abrasion characteristics.

### SUMMARY OF THE INVENTION

According to the invention there is provided a dentifrice composition comprising an abrasive system comprising a combination of at least one abrasive amorphous silica having a Radioactive Dental Abrasion (RDA) in the range 30 to 300, an oil absorption in the range 40 to 150 cm³/100 g and a weight mean particle size in the range 3 to 15 μm and a crystalline aluminosilicate having an average crystallite size below 0.2 μm plus an orally acceptable carrier.

### DETAILED DESCRIPTION OF THE INVENTION

RDA values for the abrasive system of the invention and components thereof are measured using an aqueous slurry of the component as defined hereinafter. If however the RDA were measured on the complete dentifrice composition i.e. including any optional components as defined hereinafter, the RDA values obtained may be significantly different. For example the RDA of a toothpaste formulation incorporating the materials of the invention would be in the range 25-200, preferably 30-180, more preferably 50-150.

The amount(s) of abrasive silica(s) will depend on the abrasiveness of the silicas employed. Usually the abrasive silica(s) content will be in the range of 7 to 85%, e.g. 12 to 80%, by weight of the combined silica abrasive/aluminosilicate content of the system.

The silica may be an abrasive amorphous silica (herein referred to as silica A) having a Radioactive Dental Abrasion (RDA) in the range 30 to 150, an oil absorption in the range 60 to 140 cm³/100 g and a weight mean particle size in the range 5 to 15 μm and/or an abrasive amorphous silica (herein referred to as silica B) having a Radioactive Dental Abrasion (RDA) in the range 100 to 300, an oil absorption in the range 40 to 150 cm³/100 g and a weight mean particle size in the range 3 to 15 μm.

When both silicas are employed, the quantity by weight of silica A is usually greater than that of silica B and the RDA of silica B is greater than that of silica A.

Where both silica A and silica B are present, silica B functions as a “cleaning booster” while silica A, relative to silica B, constitutes the principal silica content of the abrasive system.

In addition to at least one abrasive silica, the abrasive system of the present invention may also include a thickening silica having an RDA less than 30.

Typically the silica(s) employed in the abrasive system is/are precipitated silicas.

The components of the abrasive system of the invention are preferably in the dry state to ensure a free flowing powder with no microbial and preservation issues associated with filter cakes with high water content. The physical water content as measured by loss at 105° C. associated with the system and/or its individual components is preferably less than 20% of the system or individual component.

Generally, silica A has a low to medium RDA. Typically its RDA is at least 40, more usually at least 50. Typically its RDA is no greater than 130, e.g. no greater than 110.

The oil absorption of the silicas employed in the present invention are measured according to the test described hereinafter. The preferred range for silica A is 80 to 120 cm³/100 g. Preferably, silica A has a weight mean particle size in the range 6 to 12 μm, the size being measured by a Malvern Mastersizer®, as described hereinafter. Usually the desired particle size of silica A is obtained by subjecting the silica to a milling step.

Generally, silica B has a medium to high RDA. Typically its RDA is at least 100, more usually at least 120, e.g. at least 130. Typically its RDA is no greater than 220, more usually no greater than 200, e.g. no greater than 180.

The oil absorption of silica B is typically at least 50, more usually at least 60, e.g. at least 65 cm³/100 g. The oil absorption of silica B is typically at most 130, more usually at most 120, e.g. at most 100 cm³/100 g. Where both silica A and B are present in the system, usually silica B will have the lower oil absorption.

Preferably, silica B has a weight mean particle size of at least 2 μm, more usually at least 3 μm. Preferably, silica B has a weight mean particle size of at most 8 μm, more usually at most 6 μm, as measured by a Malvern Mastersizer®. Usually the desired particle size of silica B is obtained by subjecting the silica to a micronising comminution step.

The pHs of the silicas A and B (and any other silica present, such as a thickening silica), measured as a 5% by weight suspension, are typically at most 8, more usually at most 7.5, e.g. at most 7.0. Typically the pHs of silicas A and B (and any other silica present in the system) are at least 3.0 and more usually at least 4.0. The pH of the abrasive system is a particularly effective way of controlling the pH of the dentifrice composition employing the system. According to a feature of the invention, the components of the abrasive system are selected in such a way that the pH of the system is at most 10.0, more usually at most 9.5, e.g. at most 9.0. Such pH control may be affected by the silica content (silica A and/or B and any other silica present) of the system without the need for adjusting the inherent, high pH of the crystalline aluminosilicates by ion exchange techniques. However, we do not exclude the possibility of at least part of the crystalline aluminosilicate content being constituted by a pH-adjusted crystalline aluminosilicate, especially if an even lower toothpaste pH is required.

The amount of water present on the amorphous silicas A or B, as measured by the ignition loss at 1000° C., is usually up to 25 per cent by weight and preferably up to 15 per cent by weight. Usually the ignition loss at 1000° C. is more than 4 per cent by weight.

Crystalline aluminosilicates useful in this invention can be represented by the formula: M_{2/n}O Al₂O₃xSiO₂yH₂O wherein M represents a metal moiety, said metal having a valency of n, x indicates the molar ratio of silica to alumina and y indicates the ratio of molecules of water to alumina.

The structure and characteristics of many crystalline aluminosilicates (zeolites) are described in the standard work “Zeolite Molecular Sieves” by Donald W. Breck, published by Robert E. Krieger Publishing Company. Usually, the value of x in the above empirical formula is in the range 1.5 to 10. The value of y, which represents the amount of water contained in the voids of the zeolite, can vary widely. In anhydrous material y=0 and, in fully hydrated zeolites, y is typically up to 5.

Zeolites useful in this invention may be based on naturally-occurring or synthetic aluminosilicates but a preferred form of zeolite has the structure known as zeolite P. Particularly preferred forms of zeolite are those disclosed in EP-A-0 384 070, EP-A-0 565 364, EP-A-0 697 010, EP-A-0 742 780, WO-A-96/14270, WO-A-96/34828 and WO-A-97/06102, the entire contents of which are incorporated herein by this reference. The zeolite P described in EP-A-0 384 070 has the empirical formula given above in which M represents an alkali metal and x has a value up to 2.66, preferably in the range 1.8 to 2.66, and has a structure which is particularly useful in the present invention. More preferably, x has a value in the range 1.8 to 2.4. The zeolite P disclosed in the above patent literature is readily amenable to being produced with crystallite sizes well below 0.2 μm and agglomerate sizes (i.e. weight mean particle sizes) below 2.5 μm, even when dried to a moisture content below 20% by weight. This contrasts with other zeolites which, on drying, tend to agglomerate to large weight mean particle sizes.

The average crystallite size of the crystalline aluminosilicate, measured using the test described hereinafter is preferably between 0.01 and 0.1 μm (typically less than 0.1 μm) and, more preferably 0.02 to 0.08 μm.

The RDA of the crystalline aluminosilicate should be relatively low and is preferably less than 120, more preferably less than 100. Its RDA will usually be in excess of 30.

Additionally, preferred aluminosilicates produce minimal scratching on dental surfaces when used. Scratching can be assessed using the PAV test described hereinafter and preferred aluminosilicates have a PAV of 4 to 11, preferably 4 to 9 and more preferably 4 to 7.

The aluminosilicate preferably has a calcium binding capacity, as hereinafter defined, of at least 100 mg CaO per gram of anhydrous aluminosilicate, preferably at least 130 mg CaO per gram of anhydrous aluminosilicate and most preferably at least 150 mg CaO per gram of anhydrous aluminosilicate.

The aluminosilicate preferably has an oil absorption of at least 40 cm³/100 g and preferably in the range 40 to 100 cm³/100 g.

The aluminosilicate preferably has a weight mean particle size as measured by Malvern Mastersizer®, of at least 0.5 μm, more usually at least 1.0 μm, e.g. at least 1.8 μm. The aluminosilicate preferably has a weight mean particle size as measured by Malvern Mastersizer®, of at most 10.0 μm, more usually at most 5.0 μm, e.g. at most 3.0 μm. A most preferred range for the aluminosilicate is from 2.0 to 2.5 μm.

Usually, the preferred form of zeolite P is one in which M in the above formula consists of alkali metal ions. However, suitable forms of zeolite P include those wherein a proportion of the alkali metal moieties M has been exchanged for other metal moieties, for instance as disclosed in published International Patent Application No. WO 01/94512. Partially exchanged zeolites are particularly useful when it is desired to control the pH of the abrasive system. Such pH adjustment step involves additional processing of the zeolite and associated cost. For this reason, as mentioned above, it is preferred to buffer the effect of the high pH zeolite by means of the silica content of the abrasive system and the inherent pH of the selected silica(s).

The pH of the aluminosilicate used in the abrasive system of the invention, particularly when not partially exchanged as discussed above, is usually in excess of 10. Where the aluminosilicate present in the system is one which has undergone such ion exchange, its pH will usually be no greater than 10.

The proportions of silica and aluminosilicate present in the dental abrasive system of the invention can be varied in order to achieve a balance of properties suitable for the dentifrice composition in which it is used.

Where silica A is the only silica abrasive present in the abrasive system of the invention, it typically comprises at least 15%, more usually at least 20%, e.g. at least 30%, by weight of the combined silica abrasive/aluminosilicate content of the system. In this instance, silica A typically comprises at most 85%, more usually at most 80%, e.g. at most 70%, by weight of the combined silica abrasive/aluminosilicate content of the system. A typical range for the silica A content is between 40 and 60% by weight of the combined silica abrasive/aluminosilicate content of the system.

Where silica B is the only silica abrasive present in the abrasive system of the invention, it typically comprises at least 1%, more usually at least 5%, e.g. at least 10%, by weight of the combined silica abrasive/aluminosilicate content of the system. In this instance, silica B typically comprises at most 70%, more usually at most 50%, e.g. at most 40%, by weight of the combined silica abrasive/aluminosilicate content of the system. A typical range for the silica B content is between 15 and 35% by weight of the combined silica abrasive/aluminosilicate content of the system.

Where both silica A and silica B abrasives are present in the abrasive system of the invention, silica A typically comprises at least 25%, more usually at least 35%, e.g. at least 40%, by weight of the combined silica A/silicaB/aluminosilicate content of the system. Silica B typically comprises at least 2%, more usually at least 4%, e.g. at least 10%, by weight of the combined total silica A/silicaB/aluminosilicate. In this instance, silicas A and B typically comprise at most 80%, more usually at most 70%, e.g. at most 65%, by weight of the combined silica abrasive/aluminosilicate content of the system.

A further additional component can be a different crystalline aluminosilicate, e.g. an A, X or Y type zeolite, which acts as a cleaning booster (hereinafter referred to as “booster zeolite”). When present, the amount of booster zeolite present will usually be less than that of the crystalline aluminosilicate referred to hereinbefore (the “principal” zeolite). Usually, where a booster zeolite is employed, it is not necessary to include silica B in the abrasive system.

The booster zeolite preferably has an RDA in the range 100 to 300 and more preferably in the range 100 to 250. The PAV of the booster zeolite is preferably in the range 9 to 25 and more preferably in the range 9 to 20. The values for both the RDA and the PAV of the booster zeolite will be greater than those for the principal zeolite. The preferred oil absorption of the booster zeolite is in the range 30 to 100 cm³/100 g, more preferably 30 to 50 cm³/100 g. The weight mean particle size of the booster zeolite is preferably in the range 2.0 to 5.0 μm. The booster zeolite preferably has an average crystallite size above 0.2 μm and most preferably above 1.0 μm.

The proportions of silica A and silica B or booster zeolite present in the dental abrasive system of the invention can be varied to provide optimum cleaning with controlled abrasion. Generally, in one embodiment the proportion of silica A to booster particles by weight is in the range 100:0 to 0:100. In another embodiment the ratio is in the range 90:10 to 50:50. The term “booster particles”, as used herein refers to booster silica, B, booster zeolite or a combination of booster silica and booster zeolite.

When a dentifrice composition is prepared using the abrasive system of this invention, the components of the system may be mixed prior to combining the subsequent mixture with the other components of the dentifrice composition or may be separately added to the other components of the dentifrice composition. In each instance, the components or mixture thereof will, at least prior to combining the same with other components of the dentifrice composition, usually be in the form of a substantially dry free flowing particulate material.

A dentifrice composition containing the abrasive system according to the present invention may also include a fluoride ion source as protection against demineralisation by bacteria (caries) and/or acidic components of the diet (erosion). The fluoride ion source may be provided by any of the compounds conventionally used in toothpastes for these purposes, e.g. sodium fluoride, alkali metal monofluorophosphate, stannous fluoride and the like, with an alkali metal monofluorophosphate such as sodium monofluorophosphate being preferred. The fluoride ion source serves in a known manner for caries protection. Preferably, the fluoride ion source will be used in an amount to provide a safe yet effective amount to provide an anti-caries and anti-erosion benefit such as an amount sufficient to provide from about 25 ppm to about 3500 ppm, preferably about 1100 ppm, as fluoride ion. For example the formulation may contain 0.1-0.5 wt % of an alkali metal fluoride such as sodium fluoride.

Preferably the pH of the dentifrice composition incorporating an abrasive system of the present invention is from about 6 to 10.5, more preferably from about 7 to about 9.5. Typically the composition may contain sodium hydroxide, e.g. up to 1.0 wt. % or more to provide a suitable pH.

The abrasive system of the present invention may be incorporated in an orally acceptable carrier to produce a dentifrice composition. The term “orally acceptable carrier” means a suitable vehicle which can be used to apply the resulting dentifrice composition to the oral cavity in a safe and effective manner.

In compositions containing an abrasive system in accordance with the present invention which are usable in the manner of conventional toothpastes, i.e. which can be extruded onto a toothbrush, the orally acceptable vehicle may be of a generally conventional composition e.g. comprising a thickening agent, a binding agent and a humectant. Preferred binding agents include for example natural and synthetic gums such as xanthan gums, carageenans, alginates, cellulose ethers and esters. Preferred humectants include glycerin, sorbitol, propylene glycol and polyethylene glycol. A preferred humectant system consists of glycerin, sorbitol and polyethylene glycol.

In addition, the orally acceptable vehicle may optionally comprise one or more surfactants, sweetening agent, flavouring agent, anticaries agent (in addition to the fluoride ion source), anti-plaque agent, anti-bacterial agent such as triclosan or cetyl pyridinium chloride, tooth desensitizing agent such as potassium or strontium salts such as potassium nitrate or strontium chloride, colouring agents and pigment. Useful surfactants include the water-soluble salts of alkyl sulphates having from 10 to 18 carbon atoms in the alkyl moiety, such as sodium lauryl sulphate, but other anionic surfactants as well as non-ionic, zwitterionic and cationic surfactants may also be used.

If an aqueous orally acceptable vehicle is employed, the dentifrice composition suitably contains from about 10 to about 80 wt % humectant such as sorbitol, glycerin, polyethylene glycol or xylitol; from about 0.25 to about 5 wt % detergent; from 0 to about 2 wt % sweetener; from 0 to about 2 wt % flavouring agents; together with water and an effective amount of binding and thickening agents, such as from about 0.1 to about 15 wt %, to provide the toothpaste of the invention with the desired stability and flow characteristics.

As previously stated, the abrasive systems of the invention are capable of providing dentifrice compositions with good cleaning and are well within the abrasion limits generally considered as acceptable for commercial formulations. The cleaning ability of a composition can be assessed by the test known as the FT₁₀₀ Cleaning test, details of which are given below. The abrasive systems are tested in a dentifrice composition having a standard formulation. Preferred abrasive systems of this invention have an FT₁₀₀ Cleaning value of at least 50 per cent, preferably at least 65 per cent and most preferably above 75 per cent.

The tests used to characterise the components of the abrasive system of this invention are as follows.

Radioactive Dentine Abrasion Test (RDA)

The procedure follows the method for assessment of dentifrice abrasivity recommended by the American Dental Association (Journal of Dental Research 55(4) 563, 1976). In this procedure, extracted human teeth are irradiated with a neutron flux and subjected to a standard brushing regime. The radioactive phosphorus 32 removed from the dentin in the roots is used as the index of the abrasion of the dentifrice tested. A reference slurry containing 10 g of calcium pyrophosphate in 50 cm³ of 0.5% aqueous solution of sodium carboxymethyl cellulose is also measured and the RDA of this mixture is arbitrarily taken as 100. In order to measure a powder RDA for the precipitated silica or crystalline aluminosilicate a suspension of 10.0 g of the silica or aluminosilicate in 50 cm³ of 0.5% aqueous solution of sodium carboxymethyl cellulose is prepared and the suspension is submitted to the same brushing regime. In order to measure an RDA value for a dentifrice composition containing an abrasive system of the invention, a test slurry is prepared from 25 g dentifrice composition and 40 cm³ of water and this slurry is submitted to the same brushing regime.

Plastics Abrasion Value (PAV)

This test is based upon a toothbrush head brushing a Perspex® plate in contact with a suspension of the aluminosilicate in a sorbitol/glycerol mixture. Perspex® has a similar hardness to dentine. Therefore, a substance which produces scratches on Perspex® is likely to produce a similar amount of scratching on dentine. Normally the slurry concentration is as follows:

| | |
|---|---|
| Aluminosilicate | 2.5 g |
| Glycerol | 10.0 g |
| Sorbitol Syrup* | 23.0 g |

*Syrup contains 70% sorbitol/30% water

All components are weighed into a beaker and dispersed for 2 minutes at 1500 rpm using a simple stirrer. A 110 mm×55 mm×3 mm sheet of standard PERSPEX clear cast acrylic sheet, grade 000, manufactured by Lucite International UK Ltd, PO Box 34, Darwen, Lancashire, UK, is used for the test.

The test is carried out using a modified Wet Scrub Abrasion Tester produced by Sheen Instruments. The modification is to change the holder so that a toothbrush can be used in place of a paintbrush. In addition, a weight of 400 g is attached to the brush assembly, which weighs 145 g, to force the brush onto the PERSPEX sheet. The toothbrush has a multi-tufted, flat trim nylon head with round ended filaments and medium texture, for example, the well-known Professional Mentadent P gum health design, or an equivalent toothbrush. A galvanometer is calibrated using a 45° Plaspec gloss head detector and a standard (50% gloss) reflecting plate. The galvanometer reading is adjusted to a value of 50 under these conditions. The reading of the fresh PERSPEX sheet is then carried out using the same reflectance arrangement.

The fresh piece of PERSPEX sheet is then fitted into a holder. 2 cm³ of the dispersed aluminosilicate, sufficient to lubricate fully the brushing stroke, is placed on the sheet and the brush head is lowered onto the sheet. The machine is switched on and the sheet is subjected to 300 strokes of the weighted brush head. The sheet is removed from the holder and all the suspension is washed off. It is then dried and its gloss value is determined again. The abrasion value is the difference between the unabraded gloss value and the gloss value after abrasion. This test procedure, when applied to known abrasives, gave the following typical values.

| | PAV |
|---|---|
| Calcium carbonate (15 μm) | 32 |
| Silica xerogel (10 μm) prepared according to | 25 |
| GB 1 262 292 | |
| Alumina trihydrate (Gibbsite) (15 μm) | 16 |
| Calcium pyrophosphate (10 μm) | 14 |
| Dicalcium phosphate dihydrate (15 μm) | 7 |

Oil Absorption

The oil absorption is determined by the ASTM spatula rub-out method (American Society of Test Material Standards D 281). The test is based on the principle of mixing linseed oil with the silica or aluminosilicate by rubbing with a spatula on a smooth surface until a stiff putty-like paste is formed which will not break or separate when it is cut with a spatula. The oil absorption is then calculated from the volume of oil (V cm³) used to achieve this condition and the weight, W, in grams, of silica or aluminosilicate by means of the equation:
Oil absorption=(V×100)/W, i.e. expressed in terms of cm³ oil/100 g silica or aluminosilicate.
Weight Mean Particle Size by Malvern Mastersizer®

The weight mean particle size of the silica or aluminosilicate is determined using a Malvern Mastersizer® model S, with a 300 RF lens and MS17 sample presentation unit. This instrument, made by Malvern Instruments, Malvern, Worcestershire, uses the principle of Fraunhofer diffraction, utilising a low power He/Ne laser. Before measurement, the sample is dispersed ultrasonically in water for 5 minutes (in the case of silica) and 30 seconds (in the case of aluminosilicate) to form an aqueous suspension. The Malvern Mastersizer® measures the weight particle size distribution of the silica or aluminosilicate. The weight mean particle size (d₅₀) or 50 percentile and the percentage of material below any specified size are easily obtained from the data generated by the instrument.

Average Crystallite Size of Aluminosilicate

The average crystallite size is determined from photographs made in a scanning electron microscope. The crystalline aluminosilicate is dried to a water content of about 1 to 3 weight per cent and the agglomerates are broken up with a pestle and mortar. From the photographs, a sufficient number of crystals, e.g. 100, is counted and their size measured to determine a statistically significant average (arithmetical mean) size.

Calcium Binding Capacity of Aluminosilicate

The aluminosilicate is first equilibrated to constant weight over saturated sodium chloride solution and the water content is measured. An amount is dispersed in 1 cm³ water in an amount corresponding to 1 g dm⁻³ (dry weight) and the resulting dispersion is injected into a stirred solution of total volume 54.923 cm³, consisting of 0.01M NaCl solution (50 cm³) and 0.05M CaCl₂ (3.923 cm³). This corresponds to a concentration of 200 mg of CaO per dm³, i.e. just greater than the theoretical maximum amount (197 mg) that can be taken up by an aluminosilicate of Si:Al ratio 1.00. The dispersion is vigorously stirred at a temperature of 25° C. for 15 minutes, after which time the Ca²⁺ ion concentration is determined using a calcium electrode. The Ca²⁺ ion concentration measured is subtracted from the initial concentration to give the effective calcium binding capacity of the aluminosilicate sample

FT₁₀₀ Cleaning Test

The test is fully described in “Dental stain prevention by abrasive toothpastes: A new in vitro test and its correlation with clinical observations”, P. L. Dawson et al., J. Cosmet. Sci., 49, 275-283 (1998). The abrasive system to be tested is incorporated into the following Dentifrice Formulation 1.

Dentifrice Formulation 1

| INGREDIENT | % by wt. |
|---|---|
| Sorbitol, 70% Soln. | 26.00 |
| Glycerol, 98% min. solution | 10.00 |
| Polyethylene glycol (PEG 6) | 3.00 |
| Principal Amorphous silica | A |
| Booster Particles | B |
| Crystalline aluminosilicate | C |
| Silica thickener | D |
| Sodium lauryl sulphate | 1.15 |
| Titanium Dioxide | 1.45 |
| Xanthan gum | 0.7 |
| NaF | 0.24 |
| Saccharin | 0.23 |
| Flavor | 1 |
| De-ionised water | To 100 |
| Total | 100.00 |

The quantities A, B and C are determined by the abrasive system under test. The quantity of thickening silica (“D”) is adjusted to ensure that the cohesion of the paste, as measured by the toothpaste cohesion test defined hereinafter is in the range 150 to 430 g.
Substrate

A substrate consisting of highly polished 17 mm sintered, pure hydroxyapatite (HAP) discs is prepared. The discs are polished using a Buehler rotary grinder and P600 wet paper, followed by P1200 lapping paper to give a mirror-like finish to simulate enamel tooth surface. The whiteness of the discs (using the CIE 1976 L*a*b* system) before cleaning, L* (clean), is then measured using a Minolta Chroma-meter CR200, which has been calibrated against a standard calibration tile.

Staining

A fresh staining solution is prepared by mixing 50g of a 0.5% by weight solution of tannic acid and 50 g of a 0.5% by weight solution of ammonium ferric sulphate to form a fresh colloidal iron (III) tannic acid complex (“ferric tannate”), which has a dark colour. The fresh mixture is painted onto the HAP discs using a fine squirrel-hair brush and gently dried with a warm hairdryer. A sufficient number of coats of staining solution are applied in order to produce a darkness measurement of L*=50+/−5 as determined using a Minolta Chroma-meter CR200. This value is designated L* (soiled)

Toothpaste Slurry Preparation

A diluent is prepared, which consists of:

| | % by weight |
|---|---|
| Sodium carboxymethyl cellulose (SCMC 7M) | 0.5 |
| Glycerol | 5.0 |
| Formalin | 0.1 |
| Demineralised Water | 94.4 |

The toothpaste under test is weighed into a plastic beaker and mixed with diluent and demineralised water in the following proportions by weight:
Toothpaste 33.3%; Diluent 33.3%; Water 33.3% to produce a 100 g toothpaste slurry preparation, which is mixed for one minute with a high shear Heidolph mixer at 4000 r.p.m.
The toothpaste slurry is prepared immediately prior to carrying out the test to avoid any chances of settlement.
   Brushing

The stained HAP discs are then mounted horizontally in the bottom of a trough containing the toothpaste slurry under test and 263 g weighted Mentadent P Professional soft-nylon flat trim toothbrush heads are oscillated over the disc surfaces using a mechanical scrubbing machine (modified Martindale abrasion tester). An oscillation rate of 150 cycles per minute is used. The toothbrush heads are 34-tuft flat-trim 0.2 mm bristle nylon heads and are weighted via weights loaded onto vertical spindles mounted in linear ball bearings. For the FT₁₀₀ test soil removal after 100 oscillations is monitored. The whiteness of the HAP discs after cleaning, L* (cleaned) is measured using a Minolta Chroma-meter CR200. The Cleaning is defined as the % FT₁₀₀ Removal where

$$\% {FT}_{100} Removal = \frac{{L}^{*} ( cleaned ) - {L}^{*} ( soiled )}{{L}^{*} ( clean ) - {L}^{*} ( soiled )} \times 100$$

Toothpaste Cohesion

The cohesion of a toothpaste is a good measure of the “stand-up” properties of the ribbon when it has been extruded from a toothpaste tube onto a toothbrush. Higher cohesion values indicate firmer toothpaste ribbons, whereas low cohesion numbers are obtained from low viscosity, poorly structured toothpastes, which quickly sag into the bristles of the brush. It is generally required that a dentifrice has a cohesion within the range of 150-430 g to provide a good quality, extrudable ribbon, which does not sag and is not too firm.

The basic principle of the test is to measure the weight in grams required to pull two parallel plates apart, which have a specific layer of toothpaste sandwiched between them. The purpose built equipment consists of:
1) A spring balance in which the spring can be extended from 0-430 g in 100 mm of length. The spring has a calibration scale of zero to 430 g in 10 g intervals and can be adjusted to zero at the start of the test.
2) A motor driven ratchet, which is attached to the bottom plate and can be used to apply a constant, uniform, smooth vertical pull on the bottom plate of 5 cm per minute.
3) An upper polished chrome circular plate of 64 mm diameter, which has a hook on the upper side that can be attached to the spring balance. The polished plate has three small identical spacer pieces of polished chrome on the underside of the plate, as an integral part of the plate. These protrude to a depth of 4 mm, which determines the toothpaste film thickness when the equipment is assembled to carry out the test.
4) A lower polished chrome circular plate of 76 mm diameter, which is attached underneath to a motor driven ratchet. Two short pegs are located on the top of the plate so that the top plate can be positioned on the bottom plate concentrically from the centres.
5) A metal framework which allows the top plate to be situated concentrically above the bottom plate and the bottom plate to be adjusted so that the plate is approximately horizontal (achieved through the use of levelling feet on the base of the equipment).

15-20 g of toothpaste is evenly distributed onto the underside of the upper plate and the plate is carefully positioned onto the top of the bottom plate, using the two short pegs to locate the edge of the top plate. The top plate is firmly pressed down onto the bottom plate, until all three spacers have made contact with the bottom plate. Excess toothpaste, which has been squeezed out from between the two plates is then removed with a spatula, such that no toothpaste extends beyond the diameter of the top plate. The upper plate is then connected to the spring balance and the scale set to zero grams. The equipment is then switched on to allow the motor driven ratchet to lower the bottom plate. The spring is gradually extended and the highest observed weight is noted, as the two parallel plates sandwiched with toothpaste are eventually pulled apart. This is the toothpaste cohesion recorded in grams.

pH

This measurement is carried out on a 5 weight per cent suspension of the silica and/or aluminosilicate in boiled demineralised water (CO₂ free).

Ignition Loss at 1000° C.

Ignition loss is determined by the loss in weight of a silica when ignited in a furnace at 1000° C. to constant weight.

Moisture Loss at 105° C.

Moisture loss is determined by the loss in weight of a silica and/or aluminosilicate when heated in an oven at 105° C. to constant weight.

The invention is illustrated by the following non-limiting examples.

### EXAMPLES

In order to demonstrate the use of the invention, the aforementioned Dentifrice Formulation 1 was used as a base formulation in which particle components A, B, C and D were varied according to the following examples:

### Example 1

14% by weight Sorbosil AC35 was used as the principal amorphous silica abrasive, A, and 10% by weight Doucil A24 Zeolite was used as the crystalline aluminosilicate, C, in dentifrice formulation 1, together with 6.5% by weight thickening silica, D having a pH of 6.4. There were no booster particles, B, in this example. The pH of the mixture comprising abrasive A, zeolite C and thickener D was 8.5. The properties of the cleansing particles used are given in Tables 1 and 2.

The properties of the resultant toothpaste are given in Table 3

### Example 2

14% by weight Sorbosil AC35 was used as the principal amorphous silica abrasive, A, and 20% by weight Doucil A24 Zeolite was used as the crystalline aluminosilicate, C, in dentifrice formulation 1, together with 4% by weight thickening silica, D having a pH of 6.4. There were no booster particles, B, in this example. The pH of the mixture comprising abrasive A, zeolite C and thickener D was 9.0. The properties of the cleansing particles used are given in Tables 1 and 2.

The properties of the resultant toothpaste are given in Table 3

### Example 3

14% by weight Sorbosil AC35 was used as the principal amorphous silica abrasive, A, and 10% by weight Doucil A24 Zeolite was used as the crystalline aluminosilicate, C, in dentifrice formulation 1, together with 5% by weight thickening silica, D having a pH of 6.4. 4.2% by weight of Sorbosil AC43 silica booster particles, B, was also added to the formulation. The pH of the mixture comprising abrasive A, booster B, zeolite C and thickener D was 8.2. The properties of the cleansing particles used are given in Tables 1 and 2. The properties of the resultant toothpaste are given in Table 3.

TABLE 1
Zeolite Powder Properties

| | | | | Weight | Average | | Calcium |
|---|---|---|---|---|---|---|---|
| | | | | mean | Crys- | | binding |
| | Pow- | | Oil | particle | tallite | | capacity |
| Particle | der | | absorption | size | size | | mg |
| type | RDA | PAV | (cm³/100 g) | (μm) | (μm) | pH | CaO/g |
| Doucil | 82 | 8 | 58 | 2.25 | 0.06 | 11.4 | 160 |
| A24 | | | | | | | |
| Zeolite | | | | | | | |

Doucil A24 Zeolite is a crystalline aluminosilicate available from INEOS Silicas Limited, Warrington, UK.

TABLE 2
Abrasive Silica Powder Properties

| | | | Weight | Ignition | |
|---|---|---|---|---|---|
| | | | mean | Loss at | |
| | Powder | Oil absorption | particle size | 1000° C. | |
| Particle type | RDA | (cm³/100 g) | (μm) | (%) | pH |
| Sorbosil AC35 | 105 | 95 | 9 | 9.5 | 6.5 |
| Sorbosil AC43 | 160 | 75 | 3.5 | 11.0 | 5.5 |

Sorbosil AC35 is a toothpaste abrasive silica (principal amorphous silica) available from INEOS Silicas Limited, Warrington, UK.
Sorbosil AC43 is a toothpaste cleaning booster silica available from INEOS Silicas Limited, Warrington, UK.

TABLE 3
Toothpaste Properties

| Example | Toothpaste | Toothpaste FT₁₀₀ |
|---|---|---|
| No | RDA | Cleaning |
| 1 | 125 | 67.1 |
| 2 | 138 | 80.5 |
| 3 | 153.5 | 79.7 |

What is claimed is:

1. An abrasive system forming part of a dentifrice composition comprising at least one abrasive amorphous silica having a Radioactive Dental Abrasion (RDA) in the range 30 to 300, an oil absorption in the range 40 to 150 cm³/100 g and a weight mean particle size in the range 3 to 15 μm and a crystalline aluminosilicate having an average crystallite size below 0.2 μm.

2. An abrasive system as claimed in claim 1 in which including an abrasive amorphous silica (herein referred to as silica A) having a Radioactive Dental Abrasion (RDA) in the range 30 to 150, an oil absorption in the range 60 to 140 cm³/100 g and a weight mean particle size in the range 5 to 15 μm and/or an abrasive amorphous silica (herein referred to as silica B) having a Radioactive Dental Abrasion (RDA) in the range 100 to 300, an oil absorption in the range 40 to 150 cm³/100 g and a weight mean particle size in the range 3 to 15 μm.

3. An abrasive system as claimed in claim 2 in which silica A has an RDA no greater than 130.

4. An abrasive system as claimed in claim 2 in which silica A has an oil absorption of 80 to 120 cm³/100 g.

5. An abrasive system as claimed in claim 2 in which silica A has a weight mean particle size of up to 6 to 12 μm.

6. An abrasive system as claimed in claim 2 in which silica B has an RDA of at least 100.

7. An abrasive system as claimed in claim 2 in which silica B has an oil absorption of 50 to 130.

8. An abrasive system as claimed in claim 2 in which both silica A and B are present in the system and silica B has the lower oil absorption.

9. An abrasive system as claimed in claim 2 in which silica B has a weight mean particle size of at least 2 μm.

10. An abrasive system as claimed in claim 2 in which the pH of silica A and/or B is at most 8.

11. An abrasive system as claimed in claim 1 in which the components of the system are selected in such a way that the pH of the system is at most 10.0.

12. An abrasive system as claimed in claim 2 in which the amount of water present on silica A and/or silica B is usually up to 25 per cent by weight.

13. An abrasive system as claimed in claim 1 in which the aluminosilicate has the formula: M_{2/n}O Al₂O₃ xSiO₂ yH₂O.

14. An abrasive system as claimed in claim 1 in which the aluminosilicate is zeolite P.

15. An abrasive system as claimed in claim 13 in which x has a value up to 2.66.

16. An abrasive system as claimed in claim 13 in which x has a value in the range 1.8 to 2.66.

17. An abrasive system as claimed in claim 13 in which x has a value in the range 1.8 to 2.4.

18. An abrasive system as claimed in claim 1 in which the aluminosilicate has a crystallite size less than 0.1 μm.

19. An abrasive system as claimed in claim 1 in which the aluminosilicate has a crystallite size between 0.02 to 0.08 μm.

20. An abrasive system as claimed in claim 1 in which the RDA of the crystalline aluminosilicate is less than 120.

21. An abrasive system as claimed in claim 1 in which the PAV of the crystalline aluminosilicate is up to 11, preferably up to 9.

22. An abrasive system as claimed in claim 1 in which the aluminosilicate has a calcium binding capacity of at least 100 mg CaO per gram of anhydrous aluminosilicate.

23. An abrasive system as claimed in claim 1 in which the aluminosilicate has a calcium binding capacity of at least 130 mg CaO per gram of anhydrous aluminosilicate.

24. An abrasive system as claimed in claim 1 in which the aluminosilicate has an oil absorption of at least 40 cm³/100 g.

25. An abrasive system as claimed in claim 1 in which the aluminosilicate has a weight mean particle size of at least 0.5 μm.

26. An abrasive system as claimed in claim 1 in which the aluminosilicate has a weight mean particle size of up to 10.0 μm.

27. An abrasive system as claimed in claim 1 in which the aluminosilicate has a weight mean particle size from 2.0 to 2.5 μm.

28. An abrasive system as claimed in claim 1 in which the crystalline aluminosilicate is derived from a zeolite P with the formula M_{2/n}O Al₂O₃ xSiO₂ yH₂O where M is an alkali metal and in which at least a proportion of the alkali metal M has been exchanged for one or more other metal moieties.

29. An abrasive system as claimed in claim 2 in which the silica A comprises at least 15% by weight of the combined silica abrasive/aluminosilicate content of the system.

30. An abrasive system as claimed in claim 2 in which the silica B comprises at least 1% by weight of the combined silica abrasive/aluminosilicate content of the system.

31. An abrasive system as claimed in claim 2 in which both silica A and silica B are present, silica A comprising at least 25% by weight of the combined silica abrasive/aluminosilicate content of the system and silica B comprising at least 2% by weight of the combined total silica A/silicaB/aluminosilicate.

32. An abrasive system as claimed in claim 2 in which both silica A and silica B are present and comprise at most 80% by weight of the combined silica abrasive/aluminosilicate content of the system.

33. An abrasive system as claimed in claim 1 further including a different crystalline aluminosilicate which acts as a cleaning booster having an RDA in the range 100 to 300.

34. An abrasive system as claimed in claim 33 in which the PAV of the booster aluminosilicate is in the range 9 to 25.

35. An abrasive system as claimed in claim 33 in which the booster aluminosilicate has an oil absorption in the range 30 to 100 cm³/100 g.

36. An abrasive system as claimed in claim 33 in which the booster aluminosilicate has a weight mean particle size in the range 2.0 to 5.0 μm.

37. An abrasive system as claimed in claim 1 in which the components of the system are in the dry state to ensure a free flowing powder.

38. An abrasive system as claimed in claim 1 including a thickening silica having an RDA less than 30.

39. A dentifrice composition comprising an abrasive system comprising a combination of at least one abrasive amorphous silica having a Radioactive Dental Abrasion (RDA) in the range 30 to 300, an oil absorption in the range 40 to 150 cm³/100 g and a weight mean particle size in the range 3 to 15 μm and a crystalline aluminosilicate having an average crystallite size below 0.2 μm plus an orally acceptable carrier.

40. A dentifrice composition as claimed in claim 39 including an abrasive amorphous silica (herein referred to as silica A) having a Radioactive Dental Abrasion (RDA) in the range 30 to 150, an oil absorption in the range 60 to 140 cm³/100 g and a weight mean particle size in the range 5 to 15 μm and/or an abrasive amorphous silica (herein referred to as silica B) having a Radioactive Dental Abrasion (RDA) in the range 100 to 300, an oil absorption in the range 40 to 150 cm³/100 g and a weight mean particle size in the range 3 to 15 μm plus an orally acceptable carrier; usually the quantity by weight of silica A being greater than that of silica B and the RDA of silica B being greater than that of silica A when both silicas are employed.

41. A dentifrice composition as claimed in claim 39 wherein the abrasive silica(s) will be in the range of 7 to 85% by weight of the combined silica abrasive/aluminosilicate content of the system.

42. A dentifrice composition as claimed in claim 41 wherein the abrasive system includes a thickening silica having an RDA less than 30.

43. A dentifrice composition as claimed in claim 1 wherein the pH is from 6 to 10.5.

44. A dentifrice composition as claimed in claim 39 in which the aluminosilicate has the formula: M_{2/n}O Al₂O₃ xSiO₂ yH₂O

45. A dentifrice composition as claimed in claim 39 in which the aluminosilicate is zeolite P.

46. A dentifrice composition as claimed in claim 39 in which the aluminosilicate has a crystallite size less than 0.1 μm.

47. A dentifrice composition as claimed in claim 46 in which the RDA of the aluminosilicate is less than 120.

48. A dentifrice composition as claimed in any of the preceding claims wherein the RDA of a complete composition is 25-200.
